(19) United States
(12) Patent Application Publication
(10) Pub. No.: US 2005/0282211 A1
(43) Pub. Date: Dec. 22, 2005

(54) Probe optimization methods

(76) Inventors: Emile F. Nuwaysir, Madison, WI (US); Roland Green, Madison, WI (US); Rebecca M. Selzer, Verona, WI (US); Todd Richmond, Madison, WI (US); Mark McCormick, Madison, WI (US); Steven Smith, Fitchburg, WI (US)

Correspondence Address: QUARLES & BRADY LLP, FIRSTAR PLAZA, ONE SOUTH PINCKNEY STREET, P.O BOX 2113 SUITE 600, MADISON, WI, 53701-2113, US

(21) Appl. No.: 11/157,629

(22) Filed: Jun. 21, 2005

Related U.S. Application Data
(60) Provisional application No. 60/581,574, filed on Jun. 21, 2004.

(51) Int. Cl. C12Q 1/68; C12P 19/34

(52) U.S. Cl. 435/6; 435/91.2

(57) ABSTRACT

The present invention provides methods for optimizing nucleic acid detection assays for use in basic research and clinical research. Specifically, the invention provides a method for empirically optimizing nucleic acid probes by testing them with samples containing genomic DNA with variations in copy number in different regions of the genome. The invention enables the optimization of probes for any hybridization based assay including microarrays, bead-based assays, genotyping assays and RNAi assays.

### CROSS-REFERENCE TO RELATED APPLICATIONS

This patent application claims priority from U.S. Provisional Patent Application Ser. No. 60/581,574 filed Jun. 21, 2004.

### STATEMENT REGARDING FEDERALLY SPONSORED RESEARCH OR DEVELOPMENT

Not Applicable.

### BACKGROUND OF THE INVENTION

The advent of DNA microarray technology makes it possible to build an array of hundreds of thousands of DNA sequences in a very small area, such as the size of a microscopic slide. See, e.g., U.S. Pat. No. 6,375,903 and U.S. Pat. No. 5,143,854, each of which is hereby incorporated by reference in its entirety. The disclosure of U.S. Pat. No. 6,375,903, also incorporated by reference in its entirety, enables the construction of so-called maskless array synthesizer (MAS) instruments in which light is used to direct synthesis of the DNA sequences, the light direction being performed using a digital micromirror device (DMD). Using an MAS instrument, the selection of DNA sequences to be constructed in the microarray is under software control so that individually customized arrays can be built to order. In general, MAS based DNA microarray synthesis technology has been optimized such that it allows for the parallel synthesis of over 800,000 unique oligonucleotides in a very small area of a standard microscope slide in a matter of a few hours. The microarrays are generally synthesized by using light to direct which oligonucleotides are synthesized at specific locations on an array, these locations being called features.

With the availability of the entire genomes of hundreds of organisms, for which a reference sequence has generally been deposited into a public data base, microarrays have been used to perform sequence analysis on DNA isolated from such organisms. Microarray methods that allow the detection of changes or variations in DNA sequence are useful for the determination of any number of conditions associated in higher eukaryotes with disease states. Another type of chromosomal variation, changes in copy number, are typically the result of amplification or deletions of stretches of chromosomes and more difficult to detect using prior microarray technology. While large amplification and deletion or translocations can be readily detected by traditional karyotyping methods, the amplification or deletion of smaller DNA fragments within a chromosome can be difficult or impossible to detect by karyotyping or any other conveniently available laboratory technique.

Techniques have recently been developed that apply microarray technology to changes in DNA copy number that have enabled progressively finer mapping of the location of amplification or deletion events. This technique is called array comparative genomic hybridization, aCGH. The ultimate resolution of microarray methods is limited only by the resolution of the probes selected (i.e. their frequency and spacing along the length of the DNA region under study). To get the best possible probe resolution using the simplest technique, one selects probes spanning the entire genome of an organism. The genome spanning probes are designed in a head to tail configuration to hybridized to overlapping portions of the genome to thus cover the entire genomic sequence. However, given the size of the genomes of most eukaryotes, this spanning technique is beyond the capacity of most DNA microarray technologies. For example, if the human genome were to be studied at this resolution with aCGH using probes of 100 bp in length, the array would still need to contain 33,000,000 probes for complete coverage of the entire human genome.

An alternative is to spread a more limited set of probes out on the array, focusing on areas of interest (for example gene coding regions) to assure complete coverage within the technical limits of the array. This subset of representative probes is more likely to report on any changes in DNA copy number if their response to changes in DNA copy number has been verified experimentally prior to their use in an aCGH setting. The empirical optimization of probes poses a technical challenge because one requires the amplification of a limited (and known) subset of genomic DNA (gDNA) in the presence of a full gDNA background to verify probe performance.

The best means of verifying that the signal intensity of a given probe is in direct response to the concentration of the complimentary DNA fragment in a population is to perform several hybridizations with varying sample concentrations of the analyte DNA and select those probes that respond appropriately. This empirical method is difficult when working with large, complex genomes such as human, mouse or rat, since most gDNA preparations are a fragmented mixture of DNA fragments from the entire genome or several genomes, all represented equally. For a given aCGH study where a particular chromosome is the focus, the ideal composition of DNA for empirical probe optimization would hold all other relative chromosomal DNA concentrations fixed and increment the concentration of chromosome of interest, in steps of one copy number, and hybridize each mixture to test arrays. Those probes that respond proportionately to changes in the test chromosome copy number are the optimized subset which are appropriate for prospective analysis of DNA copy number changes in unknown samples. Therefore, alternative methods for efficiently and accurately using microarrays to identify amplifications and deletions of smaller chromosomal DNA fragments in the genomes of organisms would be a desirable contribution to the art.

### BRIEF SUMMARY OF THE INVENTION

The present invention is summarized as methods for developing and optimizing nucleic acid detection assays for use in basic research and clinical research. In particular the invention provides a method for optimizing probes used to identify at least one genetic alteration in a test genome. The method includes providing a genomic nucleic acid sample mixture comprising a test genomic sample and a reference genomic sample, wherein the test genomic sample has genetic alterations; labeling the nucleic acids in the genomic sample mixture; hybridizing the labeled genomic sample mixture to a hybridization array, such that an intensity pattern is produced, wherein the hybridization step is performed at least one time; and selecting optimized probes corresponding to a target region in the test genome, wherein the probes exhibit a signal intensity proportionate to the copy number of the applied sample relative to the reference genomic sample. The method also includes identifying at least one genetic alteration in the test genome.

On aspect of the invention provides that the nucleic acid probes are either DNA or RNA.

Another aspect of the invention provides that the genetic alteration is an amplification or deletion in a chromosome.

In this embodiment, the genetic alteration can cover a broad region of the genome, such as an entire chromosome.

In another aspect, the invention provides a method for the optimization of probes for any hybridization based assay including microarrays, bead-based assays, genotyping assays and RNAi assays.

A further aspect of the invention is to use the method of the invention in optimizing probes used in the fields of genomics, pharmacogenomics, drug discovery, food characterization, genotyping, diagnostics, gene expression monitoring, genetic diversity profiling, RNAi, whole genome sequencing and polymorphism discovery, or any other applications involving the detection of genetic alteration involving an amplification or deletion in a chromosome.

Other objects advantages and features of the present invention will become apparent from the following specification.

### BRIEF DESCRIPTION OF THE SEVERAL VIEWS OF THE DRAWINGS

FIG. 1 is an intensity vs. chromosomal position plot showing exemplary data from a pre-optimized probe set, indicating a necessity for probe optimization resulting from a CHR7 TYR homozygous deletion in the target region.

FIG. 2 is an intensity plot of optimized probe intensities for a selected probe set vs. chromosomal position.

FIGS. 3A-B show intensity plots comparing the data from multiple hybridizations of homozygous deletion lines on the arrays for all probes and optimized probes vs. chromosomal position.

FIGS. 4A-B show intensity plots comparing the data from multiple hybridizations of heterozygous deletion lines on the arrays for all probes and optimized probes vs. chromosomal position.

### DETAILED DESCRIPTION OF THE INVENTION

The present invention relates to a method for empirically optimizing probes utilizing genomic samples of known differential copy number and composition. For example, by making multiple microarrays with multiple variations in probe design all tested against a genomic sample having a know region of amplified or deleted DNA, it then becomes possible to identify probes or probe sets which best reveal the amplified or deleted DNA.

Thus, in one embodiment, the invention provides a method for optimizing nucleic acid probes used to identify at least one genetic alteration in a test genome. The method includes providing a genomic nucleic acid sample mixture comprising a test genomic sample and a reference genomic sample, wherein the test genomic has genetic alterations and can be either DNA or RNA. The genomic sample mixture is then labeled and hybridized to a hybridization array having a variety of probes for the sequences of interest or even spanning that sequence. From testing the sample against the array an intensity pattern is produced from the hybridizations which do occur and the hybridizations vary in intensity of detected signal. Optimized nucleic acid probes corresponding to a target region in the test genome are then selected based on the detected signal, wherein the probes exhibit signal intensity proportionate to the copy number of the applied sample relative to the reference genomic sample. The probes can then be used in subsequent arrays to test for the amplified or deleted sequences.

The method also includes identifying at least one genetic alteration in the test genome. In this embodiment, the genetic alteration is an amplification or deletion in a chromosome. The amplification or deletion is detect using a microarray having probes optimized to detect just this amplified or deleted sequence. The genetic alteration can also cover a broad region of the genome, such as an entire chromosome.

In the normal terminology used to describe this technology, a microarray is a series of single stranded nucleic acid probes all tethered to a common substrate. The probes are arranged in a series of discrete locations on the substrate which are referred to as features. Each feature in intended to have a single, or sometimes two, species of probes within them. The microarrays are usually used for hybridization experiments wherein a sample of a nucleic acids is labeled and hybridized against the microarray. Information about sequences present in the sample is determined by determining which features contain probes that hybridized to the sample, as indicated by presence of the label after hybridization and washing. It is common to speak of probe design as if single probes are designed when in fact the concept is that all of the probes in a features would normally have the same sequence, i.e. be of the same design.

Specifically, the present invention describes an approach to artificially amplify known subsets of gDNA, in known amount, to provide a means of empirical probe optimization. There are two primary methods by which this can be accomplished.

In one approach used for probe optimization, individual or groups of metaphase chromosomes can be separated from the total population using recently devised methods employing fluorescence activated cell sorting (FACS) technology. These subgroups are then amplified, in vitro, using commercially available methods such as phi29 polymerase (Epicentre Technologies, Madison, Wis.) catalyzed whole genome amplification to provide a large amount of amplified gDNA derived from one or several chromosomes rather than the entire genome. Whole genome amplification tools for use with human genomic DNA (gDNA) may include REPLI-g™ technology (Molecular Staging, Inc New Haven, Conn.) or Genomiphi™ technology (Amersham Biosciences, Piscataway, N.J.).

The amplified pools are then combined with gDNA at known levels to produce known, artificial amplification levels of any desired copy number. The mixtures are hybridized in parallel with unamplified gDNA to array(s) using either individual arrays for each mixture or dye labeling each mixture with a unique fluorophore (e.g., Cy3 and Cy5). Any shifts in intensity, proportionate to the artificial amplification level in the applied mixture (relative to the unamplified control) are optimized probes.

The main advantage of this method is that any chromosomes or groups of chromosomes that can be separated by FACS can be amplified to provide a plentiful supply of material for probe optimization. The drawbacks are that not all chromosomes can be individually resolved given the current state of FAC-mediated chromosome sorting and there is some risk that the amplification steps can introduce experimental bias in copy number in those stretches of chromosomal DNA that are preferentially amplified by methods such as REPLI-g™ or Genomiphi™.

In another approach, radiation hybrids or other mapped cell lines of known DNA copy for the optimization process may be utilized to provide another empirical probe optimization method. In this method, the gDNA from cells with known chromosomal amplifications or deletions are used in a manner similar to that described hereinabove where their performance in aCGH is compared to a cell line or gDNA pool lacking amplifications. The advantage of this method is that, provided gDNA sources are plentiful, amplification by REPLI-g™ or other methods is not required, eliminating this source or experimental bias. A drawback of this approach is that it is dependent on the availability of a range of cell lines representing known copy number changes for every chromosome for which probe set optimization is desired. Another drawback is that the range of dosage control possible through artificial “spike-in” amplification mixtures is comparatively narrow via this method. Furthermore, the maximum increase in copy number for a given chromosome is limited to that produced by the cell line and only decreases in copy number can be simulated via dilution with gDNA of uniform copy number.

Despite these drawbacks, applicants believe that either of these approaches may be used to empirically optimize probes for aCGH and other array-based methods. For example, the invention can be used for standard gene expression analysis through the use of gDNA mixtures, where subsets of the genome have been manipulated to produce known changes in copy number, the application of these mixtures to arrays, specifically, NimbleGen DNA microarrays and the selection of probes that respond to the changes in copy number in the mixture applied. The method requires that the region of the genome where copy number has been altered in the mixture (whether over one or several chromosomes) correspond to a known chromosomal location in the genome and that the change in copy number be known. The corresponding array design must then cover this region as well as a region outside of the region of altered copy number for use as a reference to the optimization region. The method also requires a pool of gDNA where the copy number has not been altered in the target region or the region outside the target region. The two individual pools of gDNA are dye or hapten-labeled and hybridized to the array. Through a series of trial hybridizations, probes can be selected in the target region that exhibit a signal intensity proportionate to the copy number of the applied sample, relative to the unaltered control gDNA sample.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

The ability to optimize probes for microarray work is of critical importance to advancing the technology and increasing array capacity. There are certain current array designs which require 15 to 20 probes per gene and the values are averaged to allow the measurement of gene expression levels. Without the averaging, the signal levels of individual probes behave in a much less uniform and predictable way. If averaging could be avoided and an individual probe would suffice for an individual gene, the array capacity for gene coverage could be increased by a factor of 15 to 20. There currently does not exist a suitable computer-based method to predict which probe sequences will behave proportionately to changes in copy number. There also has not been described a method for probe optimization that allows for the testing and optimization of large probe sets covering broad regions of the genome (e.g., entire chromosomes).

The following examples are provided as further non-limiting illustrations of particular embodiments of the invention.

### EXAMPLES

Materials and Methods

Genomic DNA

Genomic DNA (gDNA) was obtained from previously BAC array mapped mouse cell lines bearing known (and identically mapped) heterozygous and homozygous deletions in mouse chromosome 7. The two mouse lines with deletions that were used in this preferred embodiment are as follows: 1) C32DSD (+/+, +/−, −/−) which encompasses the TYR gene; and 2) P12R30Lb (+/+, +/−) which is homozygous lethal and the estimated size of the deletion is 196,888 bases. Reference gDNA was obtained from normal mouse white blood cells. Although, this example uses cell lines from mouse, encompassed within the scope of this invention are gDNA from any source, including plants and animals, such as mammals, embryonic, new-born and adult humans. It is envisioned that gDNA can be obtained from recombinant genomes, stem cells, human solid tumor cell lines and tissue samples.

Amplification

For those experiments where additional gDNA was required, the deletion and normal DNA samples were amplified using the REPLI-g™ technology to amplify whole genomes (Molecular Staging, Inc New Haven, Conn.). It is understood by those skilled in the art that in addition to the methods for genome amplification described here, there are a variety of other methods that could serve the same purpose.

Labeling

In order to label the probes, gDNA was digested with methylase resistant four-base restriction enzymes such as Mnl I (New England Biolabs, Bethesda, Md.) to completion under recommended conditions. The reactions were purified by phenol:choroform extraction and precipitated with ethanol and salt. Digested gDNA was resuspended in water. Digested DNA was then combined with a random primer mixture, deoxynucleotides and buffer and denatured at 95° C. for five minutes and chilled on ice. The random primer labeling reaction was initiated by the addition of Klenow fragment of DNA polymerase I and incubation at 37° C. for 2-4 hours. Dye label was included in this reaction in the form of either dye labeled random primers (Tri-Link Biotechnologies, San Diego, Calif.) or the inclusion of Dye-labeled dNTPs available from Perkin-Elmer, Amersham Biosciences or other suppliers. In a typical experiment, the test sample from deletion or polysomy genome was dye labeled with Cy3 and the reference was labeled with Cy5. The two labeling reactions were pooled and precipitated and stored at −20° C. as a precipitated pellet until required for array hybridization. It is understood by those skilled in the art that in addition to the methods for nucleic acid labeling described here, there are a variety of other methods that could serve the same purpose.

Array Design

Nucleic acid probes (60 mers) covering a 10 megabase region spanning the previously mapped deletion in the aforementioned mouse cell lines were selected with spacing of 48 base pairs. The probes were synthesized as a NimbleGen DNA microarray as described herein the background. It is noted that the probes were of sufficient length to offer complete coverage of the 10 MB region in its entirety.

Hybridization

In general variant sequences are detected in a hybridization assay. The presence or absence of a given SNP or mutation is determined based on the ability of the DNA from the sample to hybridize to a complementary DNA molecule (e.g., a oligonucleotide probe). This can be accomplished using a variety of assays for hybridization and detection, which are readily available and well within the capabilities of a person of ordinary skill in the art.

While the present invention is not limited to a particular set of hybridization conditions, the following embodiment is most suitable.

In this preferred embodiment of the invention, three replicate hybridizations were performed under optimal hybridization conditions for aCGH probe optimization. By way of example, but not limitation, this embodiment uses buffers containing the following: 35% formamide, 5×SSC, and 0.1% (w/v) sodium dodecyl sulfate under conditions that include hybridizing under moderately non-stringent conditions at 45° C. for 16-72 hours.

Furthermore, it is envisioned that the formamide concentration may be suitably adjusted between a range of 30-45% depending on the probe length and the level of stringency desired. Also encompassed within the scope of the invention is that probe optimization can be obtained for longer probes (>>50mer), by increasing the hybridization temperature or the formamide concentration to compensate for a change in the probe length.

Additional examples of hybridization conditions are provided in several sources, including: Sambrook et al., Molecular Cloning: A Laboratory Manual (1989), 2nd Ed., Cold Spring Harbor, N.Y.; and Berger and Kimmel, “Guide to Molecular Cloning Techniques,” Methods in Enzymology, (1987), Volume 152, Academic Press, Inc., San Diego, Calif.; Young and Davis (1983) Proc. Natl. Acad. Sci. (U.S.A.) 80: 1194.

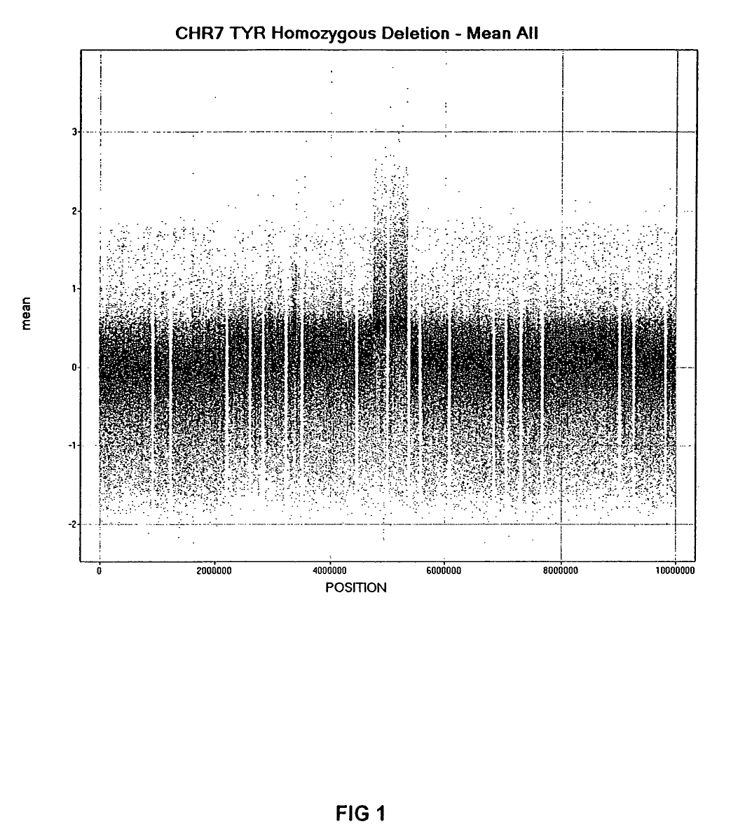

Applicants note that these conditions are designed to optimize the specificity of signal for the given probe length. The values of the three replicates were averaged and plotted as average intensity vs. chromosomal position as shown in FIGS. 1-4.

Specifically, the deletion region can be seen as a broad peak of shifted intensity centered around 5M in the plot as shown in FIG. 1. This plot also dramatically indicates the necessity for probe optimization. While the deletion region is visible, there are a large number of probes that do not accurately report the deletion in the target region and exhibit no shift signal ratio.

Optimized Probe Selection

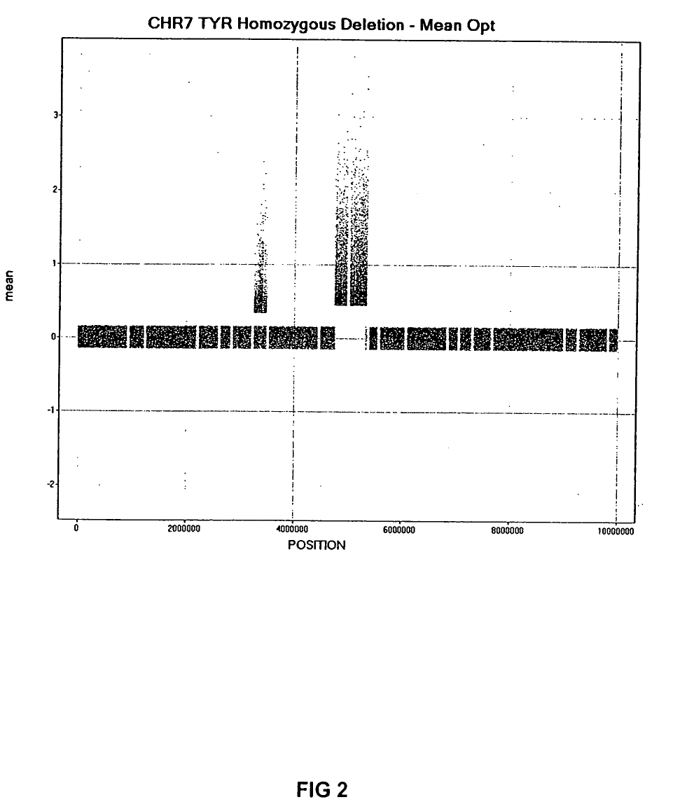

From the raw data set, those probes that accurately reflect the known copy number difference between the homozygous deletion and the reference genome are selected as an optimized subset of the entire probe set. In FIG. 2, the selected probe set is plotted vs. chromosomal position. In the example shown, additional potentially heterozygous deletion was identified and an optimized set was selected in this region.

Confirmation of Optimization Process

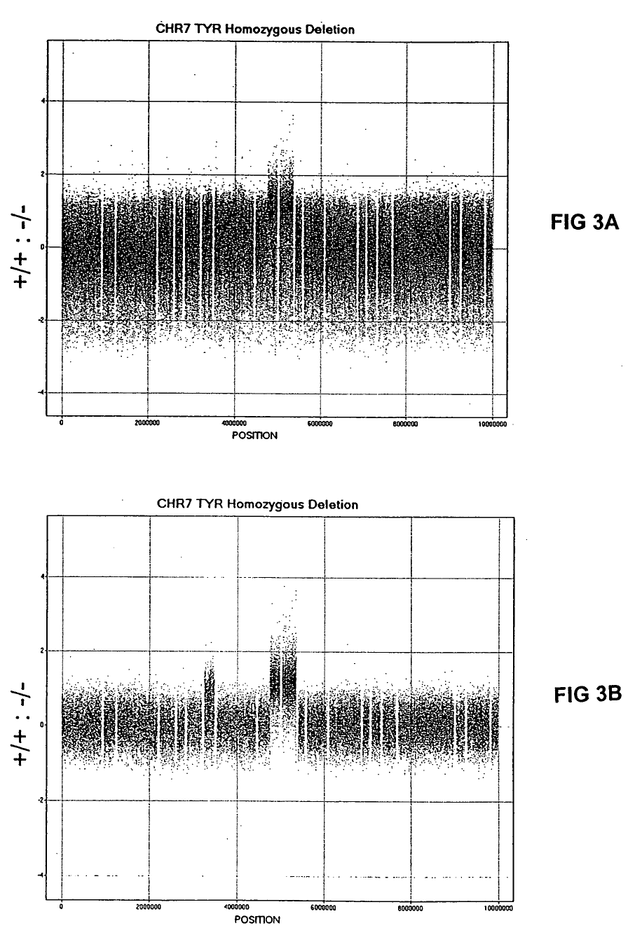

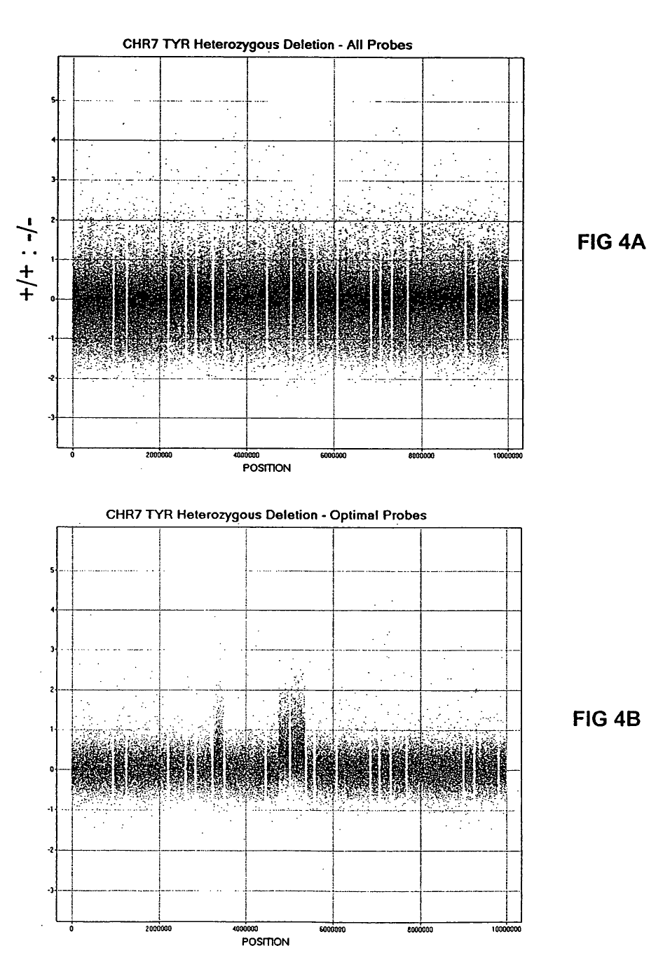

The performance of the optimized probe set was confirmed by comparing the data from multiple hybridizations of both homozygous and heterozygouse deletion lines on the arrays and plotting intensities for all probes and optimized probes vs. chromosomal position. Applicants note that in some cases, such as CHR7 TYR, the optimization process may not be strictly required for homozygous deletions as shown in FIGS. 3A-B. However, for the CHR7 TYR heterozygous deletions, the change in copy number is sufficiently subtle that detection of the target region is difficult without probe set optimization, as shown in FIGS. 4A-B.

In such cases, the present invention provides an accurate and efficient method for empirically optimizing probes by testing them with samples containing genomic DNA or RNA with variations in copy number in different regions of the genome. Therefore, in addition to optimization of probes for use in microarray-based hybridization assay, the present invention may be equally applicable for use with any hybridization based assay. Examples of hybridization assays include bead-based assays which are an essential tool for high-through put screening including DNA and single nucleotide polymorphism (SNP) assays, particularly from a multiplex perspective. Also, the present invention can be useful in genotyping assays and RNAi assays.

Furthermore, it is envisioned that by using the methods of the invention to identify aberrant regions of a genome, a map of copy-number changes of imbalances in genomes, such as complex cancer genomes can be developed. Thus, allowing the rapid identification of novel cancer genes, such as prostate, breast and other malignancies. This will enable the identification and validation of candidate biomarkers for a variety of medical conditions, as well as prognostic and diagnostic markers and druggable targets.

It is understood that certain adaptations of the invention described in this disclosure are a matter of routine optimization for those skilled in the art, and can be implemented without departing from the spirit of the invention, or the scope of the appended claims.

1. A method for optimizing nucleic acid probes used to identify at least one genetic alteration in a test genome, the method comprising the steps of:
a) providing a genomic sample mixture comprising a test genomic sample and a reference genomic sample, wherein the test genomic sample has genetic alterations;
b) labeling the genomic sample mixture;
c) hybridizing the labeled genomic sample mixture to a microarray designed to have probes that hybridize to the region of known genetic alteration, such that an intensity pattern is produced, wherein the hybridization step is performed at least one time; and
d) selecting at least one optimized probe corresponding to the region of known alteration in the test genome, wherein the probe exhibits a signal intensity which best reveals a genetic alteration in the test genomic sample relative to the reference genomic sample.

2. A method as claimed in claim 1 wherein the method also includes making subsequent microarrays including the optimized probe therein.

3. The method of claim 1 further comprising the step of:
e) identifying at least one genetic alteration in the test genome.

4. The method of claim 1 wherein the genetic alteration is an amplification or deletion in a chromosome.

5. The method of claim 1 wherein the genetic alteration covers an entire chromosome.

6. A method for testing for genomic alterations in a target genomic region, the method comprising the steps of
a) identifying optimized probes by the steps of
(1) obtaining a test genomic sample containing a genomic alteration and a reference genomic sample without the genomic alteration;
(2) making a microarray with probes designed to hybridize in the region of the genomic alteration;
(3) hybridizing the test genomic sample to the microarray and analyzing the nature of the hybridizations; and
(4) identifying at least one probe which is best optimized to reveal the genomic alteration;
b) making a microarray which contains the optimized probe;
c) hybridizing the microarray against a genomic sample of unknown content; and
d) using the information from the location of the hybridized probe to determine if the genomic alteration is in the genomic sample.

7. A method as claimed in claim 6 wherein the genomic alteration is a change in copy number of a genomic segment.

8. A method as claimed in claim 6 wherein each of the microarrays is made by a maskless array synthesis instrument.
